(19) United States
(12) Patent Application Publication
(10) Pub. No.: US 2005/0130297 A1
(43) Pub. Date: Jun. 16, 2005

(54) Cell and tissue culture device with temperature regulation

(75) Inventors: Farzin Sarem, Valbonne (FR); Leila-Ouassila Sarem Damerdji, Valbonne (FR)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Societe Nouvelle Cell Tissue Progress

(21) Appl. No.: 10/692,529

(22) Filed: Oct. 25, 2003

Related U.S. Application Data
(63) Continuation of application No. PCT/FR02/01472, filed on Apr. 26, 2002.

(30) Foreign Application Priority Data
Apr. 26, 2001 (FR) 01.05651

(51) Int. Cl. C12M 1/12; G06F 19/00; G01N 33/48; G01N 33/50

(52) U.S. Cl. 435/297.1; 702/19

(57) ABSTRACT

A device for culturing cells and tissue comprises at least one culture well (18-i), first and second reservoirs (2 and 25) each housing at least one flexible bag (6, 7; 27, 29), at least one of which is suitable for receiving a culture fluid, link means (20, 21) coupled to the wells and to the bags to allow culture fluid to flow from one reservoir to the other via the well, pressurization means (60-92) subjecting the first and second reservoirs (2 and 25) respectively to first and/or second sequences of external pressures defined by a control module (50) for the purpose of governing the flow of culture fluid in the well, and temperature regulation means (49, 51-58) controlled by the control module and arranged to maintain a first selected temperature inside the well and to subject the culture fluid leaving the first and second reservoirs in order to feed the well to a second selected temperature.

The present invention relates to the field of dynamically culturing cells and tissue using a culture fluid or nutrient medium set into motion.

The invention relates more precisely to devices for culturing cells and tissue comprising: i) one or more culture wells defining chambers for receiving cells or tissue to be cultured; ii) first and second reservoirs each housing at least one flexible bag, at least one of which is suitable for receiving a culture fluid; iii) link means coupled to the well (8) and to the bags in order to enable culture fluid to flow from one reservoir to the other via the well(s); and iv) pressurization means enabling the bags of the first and second reservoirs to be subjected respectively to first and/or second sequences of external pressures which are defined by one or more control modules and which serve to govern the flow of culture fluid in the well(s).

That type of device, as described in French patent application No. 00/00548, enables suitable flow conditions to be maintained throughout the duration of culturing. However, when the culture requires an environment that is under temperature control, devices of that type need to be placed inside a suitable incubator, thereby increasing the biological risks associated with displacements, costs, handling, and size, and makes it impossible to use a microscope to observe how the culture is progressing while incubation is taking place. In addition, the transfers lead to temperature changes that can give rise to harmful biological consequence.

An object of the invention is to provide an original solution to all or some of the above-specified drawbacks.

To this end, the invention provides a device of the type described in the introduction, in which temperature regulation means under the control of a control module are provided and serve to maintain a first selected temperature or a first selected temperature cycle within the well(s) and/or to apply a second selected temperature or a second selected temperature cycle to the culture fluid leaving at least one of the first and second reservoirs in order to feed the well(s).

Thus, temperature regulation within the device can be performed either exclusively at well level, or else exclusively at the level of the culture fluid feeding the wells, or indeed simultaneously both at well level and at the level of the culture fluid so as to minimize temperature disturbances when making exchanges between the culture fluid and the cells.

The first and second temperatures (or the first and second temperature cycles) are selected as a function of the type of culture. They can therefore be substantially identical, or else they can be different if so required by the culture. The second temperatures (or the second cycles) can also vary from one reservoir to the other should that be necessary. It is also possible to vary the temperatures (or the cycles) during the progress of culturing. To do this, parameters for causing temperatures to vary may be programmed, e.g. by being included in the program that determines the external pressure sequences imparted by the control module. Such programming may be performed using an input interface, or else directly by transferring predefined programs into a memory of the device that is coupled with (or integrated in) the control module, and then selecting one of the programs (the memory may optionally be re-writable via the above-mentioned interface).

In a first embodiment of the device of the invention, the temperature regulation means comprise a heating fluid circuit, the circuit comprising at least a first portion integrated in the walls defining the well(s) (possibly in the form of flow channels formed at the periphery of the chambers, or spaces for allowing fluid flow formed in the walls of the wells and connected to first connection means), or second and third portions integrated respectively in the walls defining the first and second reservoirs and arranged to enable a heat-conveying fluid to flow (these might be spaces formed between an inner shell and an outer shell which, once assembled together, define the first and second reservoirs), or else a combination of the first, second, and third portions. In the combination case, the second portion of the fluid circuit is preferably arranged to feed the heat-conveying fluid (liquid or gas) to the first portion, while the third portion is arranged to select the heat-conveying fluid that has flowed through the first portion. It is then particularly advantageous for the second and third portions of the fluid circuit to include second and third connection means opening out into the space between shells and suitable for being connected respectively to the first connection means and to a (main) fourth portion of the fluid circuit for feeding and collecting the heat-conveying fluid.

In this first embodiment, the heating fluid circuit preferably includes a pump coupled to a main container containing a fraction of the heat-conveying fluid (liquid or gas) and electric heater means (such as heater resistances, for example) for heating the heat-conveying fluid in controlled manner before it feeds the first, second, and third portions.

In a second embodiment of the device of the invention, the temperature regulation means comprise either first electric heater elements for providing at least a portion of the controlled heating of the well (e.g. in the form of heater resistances placed against or insulated in the walls of the wells), or else second electric heater elements for providing at least a portion of the controlled heating of the first and second reservoirs (e.g. constituted by heater resistances placed against or integrated in the walls of the reservoirs), or else a combination of the first and second electric heater elements.

Naturally, it is possible to envisage a third embodiment of the device of the invention in which the temperature regulation means comprise both a fluid circuit (as in the first embodiment) and electric heater elements (as in the second embodiment).

The device of the invention may include additional characteristics taken separately or in combination, and in particular:
each of the first and second reservoirs may comprise a top portion and a bottom portion which are interconnected by a narrow intermediate portion, the link means communicating with the bottom bags, the top portions and the bottom portions each further including a leaktight inlet. Simultaneously, pressurization means may include a fluid pump for feeding pressurization fluid at high pressure via a (main) first portion of the pressurization circuit, and a second portion of the pressurization circuit connected to top and bottom pressure-regulating valves controlled by the control module for feeding each top and bottom reservoir portion via the leaktight inlets with pressurization fluid at high pressure or at low pressure or indeed at intermediate pressure. Under such circumstances, it is particularly advantageous for the first portion of the pressurization circuit to include a sub-portion immersed in the heat-conveying fluid (liquid or gas) which is received in the main container so as to feed the second portion of the pressurization circuit with pressurization fluid that has been heated. This makes it possible to minimize temperature disturbances to the culture fluid. Furthermore, it is also possible to provide an auxiliary container housed outside the main container in contact with the heat-conveying fluid (a “bain-marie”), containing a humidifying fluid and fed with fluid under pressure by the sub-portion of the first portion of the pressurization circuit so as to feed the second portion of the pressurization circuit with a pressurization fluid presenting a selected degree of humidity. This is particularly important when the flexible bags are semi-permeable;
at least two, and preferably three or four, wells may be placed in series and communicate with one another via link means, a first well being connected to the first reservoir while a well opposite to the first is connected to the second reservoir;
the temperature regulation means may include at least one temperature sensor for providing the control module with measurements representative of the temperature inside a well, or in the immediate vicinity thereof; and
a cover may be provided to isolate the wells from the outside, and possibly also to isolate the reservoirs and indeed the entire device.

The invention also provides an installation for culturing cells and tissue and comprising at least two devices of the above-described type placed in parallel and sharing a single control unit controlling all of their control units, or itself performing their functions.

This installation may include a main fluid circuit feeding the wells and/or the reservoirs of each device in parallel. In which case, it is particularly advantageous to provide central temperature regulation means controlled by the main control unit and serving to maintain a common selected first temperature or a common selected first temperature cycle within the wells of each device, and/or to place the culture fluid which flows out from at least one of the first and second reservoirs of each device to feed its wells at a common selected second temperature or at a common selected second temperature cycle.

In a variant, the main control unit controls the temperature regulation means of each device in such a manner as to cause them to maintain a first selected temperature or a first selected temperature cycle within the wells of the corresponding device independently of one another, and/or place the culture fluid which leaves at least one of the first and second reservoirs of the corresponding device to feed its wells at a second selected temperature or a second selected temperature cycle, independently of one another.

The installation may also include a main cover for isolating the wells of each device simultaneously from the outside, possibly together with the associated reservoirs or even the complete devices.

Other characteristics and advantages of the invention appear on examining the following detailed description and from the accompanying drawings, in which;

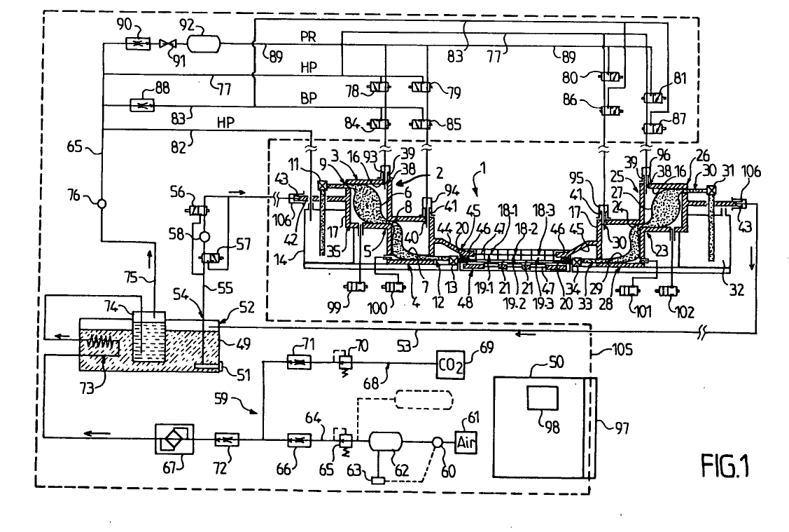

FIG. 1 is a fragmentary diagrammatic cross-section view of a culture device of the invention, having a plurality of chambers;

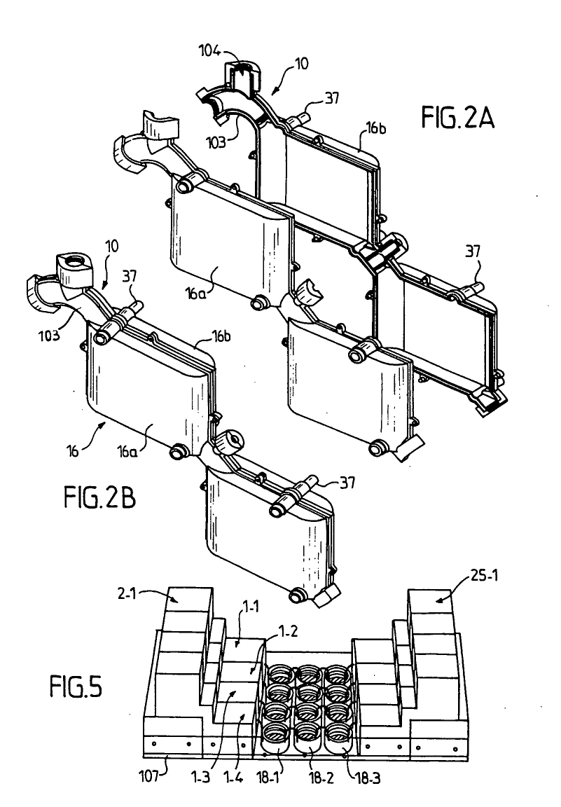

FIGS. 2A and 2B are perspective views of two inner half-shells of the reservoirs of FIG. 1, respectively before and after being assembled together;

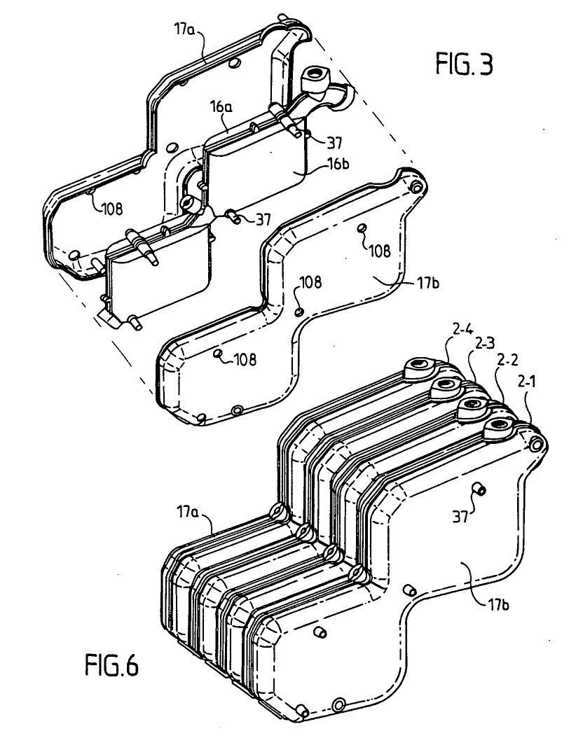

FIG. 3 is a perspective view of the two inner half-shells of FIG. 2 prior to being assembled together with two outer half-shells of the reservoirs;

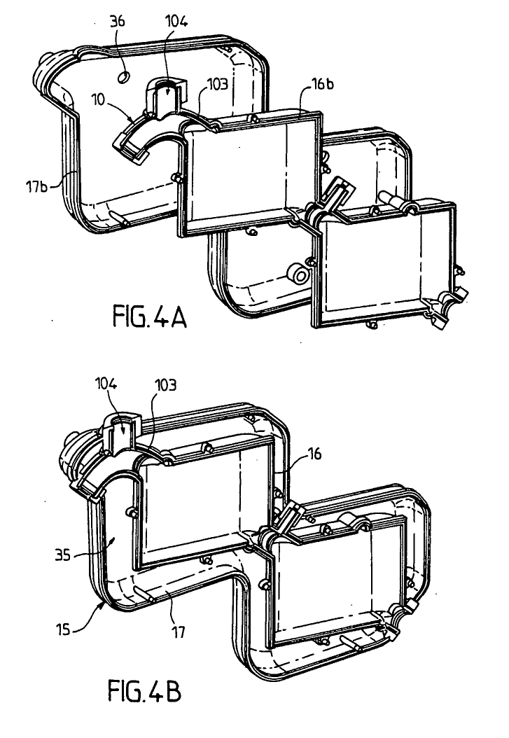

FIGS. 4A and 4B are perspective views showing how an inner half-shell is positioned inside the corresponding outer half-shell, respectively before and after assembly;

FIG. 5 is a diagrammatic perspective view of a culture instalation constituted by four culture devices placed in parallel;

FIG. 6 is a perspective view of an assembly of four double-shell reservoirs for an installation of the type shown in FIG. 5;

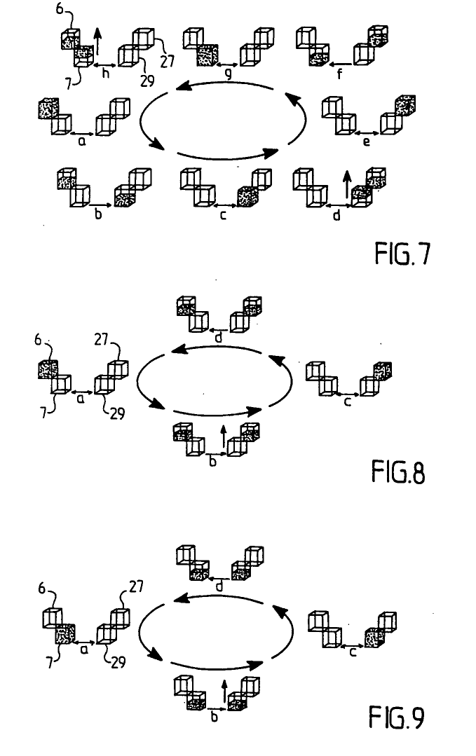

FIG. 7 is a diagram showing a sequence of eight successive go-and-return stages for culture fluid in a laminar type mode of flow;

FIG. 8 is a diagram showing a sequence of four successive go-and-return stages for culture fluid in a turbulent type mode of flow; and

FIG. 9 shows a variant of the turbulent mode shown in FIG. 8.

The accompanying drawings are in essence definitive in nature. Consequently, they can serve not only to contribute to describing the invention, but they can also contribute to defining it, where appropriate.

Reference is made initially to FIGS. 1 to 4 for describing a cell and tissue culture device in a non-limiting embodiment.

The device 1 shown in FIG. 1 comprises firstly a first reservoir 2 having a top portion 3 coupled to a bottom portion 4 via an intermediate portion 5. The reservoir 2 is defined by rigid walls 15 which give it a volume that is constant and which is discussed further below.

In the example shown, the top portion 3 of the reservoir houses a top flexible bag 6. Similarly, the bottom portion 4 houses a bottom flexible bag 7 which is connected to the top bag 6 via a duct 8 housed in the intermediate portion 5, being received closely therein so that the top and bottom portions 3 and 4 of the first reservoir 2 are isolated from each other.

The top bag 6 has an inlet/outlet 9 adapted so as to be capable of co-operating in leaktight manner with a top opening 10 formed in one of the partitions of the top portion 3 of the first reservoir 2. Thus, the top bag 6 may be connected to top access control means 11, themselves connected to a culture fluid or gas feed module, or as shown to a nutrient (or culture fluid) container 14, which is preferably pressurized. For reasons of compactness, the nutrient container 14 is placed beneath the top portion 3 of the reservoir 2, however it could be located elsewhere.

Similarly, the bottom bag 7 has an inlet/outlet 12 adapted to co-operate with a leaktight opening formed in the wall of the bottom portion 4 of the first reservoir 2, or else as shown in FIG. 1, so as to co-operate with access control means 13, in this case housed outside the bottom portion 4 of the reservoir 2.

The bottom bag 7 may comprise two substantially rigid membranes so as to prevent it from being completely flattened when it is subjected to very high pressures, since that would impede good flow of the culture fluid.

Also preferably, in the intermediate portion 5, the first reservoir 2 has an additional opening enabling a liquid or a gas to be injected into or extracted from the inside of the duct 8 either manually or automatically. The opening is preferably fitted with a septum, which is particularly suitable when the injection or extraction device is a syringe fitted with a needle. It is also preferable to provide a septum in each of the bottom and top portions of the reservoirs.

Also preferably, the top and bottom bags 6 and 7 are made of a porous material, at least in a material that is porous going from the outside towards the inside. They may be bags made of silicone, or of polydimethylsiloxane (PDSM), or indeed of polytetrafluoroethylene (PTFE), or indeed of dimethyl and methylvinyl siloxane polymers. This enables gas to be exchanged between the culture fluid which is inside the flexible bags and the gas which is trapped inside the top and bottom portions 3 and 4 of the first reservoir 2. These bags may be made of materials that are different so as to provide different functions, in particular concerning exchange with the fluid which is contained inside the reservoirs (generally the pressurization gas(es) described in greater detail below). In addition, the bags in a single reservoir may present shapes and volumes that are different.

In the example shown in FIG. 1, the bottom bag 7 communicates with a culture well 18-1 to 18-3 via the access control means 13 and the bottom opening formed in the wall of the reservoir.

The access control means 13 are preferably of the “pinch” type. They have a hollow end into which one end of link means 20 is inserted, the link means being made in the form of a duct (or tube) having its opposite end opening out into the culture chamber 19-1 of the first well 18-1. This first culture chamber 19-1 communicates with the second culture chamber 19-2 housed in the second well 18-2 via another link means 21 likewise implemented in the form a duct (or tube). Similarly, the second culture chamber 19-2 communicates with the third culture chamber 19-3 housed in the third well 18-3 via another link means 21 made in the form of a duct (or tube). Finally, in this example, a last link means 20 provides communication between the third culture chamber 19-3 and a second reservoir 25, which is described below.

The second reservoir 25 is preferably substantially identical to the first reservoir 2 as described above with reference to FIGS. 1 to 4. Consequently, in this example, it comprises a top portion 26 having a top flexible bag 27 housed therein, a bottom portion 28 having a bottom flexible bag 29 housed therein, and a narrow intermediate portion 23 housing an intermediate duct 24 coupling the top bag 27 to the bottom bag 29. This duct 24 is likewise housed narrowly in the intermediate portion 23 so that the top portion 26 is isolated in terms of gas-tightness from the bottom portion 28.

The top bag 27 has a suitable inlet/outlet 30 connected to access control means 31 which, like the access control means 11, can be connected to a gas or fluid feed device 32 or to an extractor. Similarly, the bottom bag 29 has an inlet/outlet 33 which, in the example shown, is connected to access control means 34 located in this case outside the bottom portion 28 of the second reservoir 25.

The second reservoir 25 preferably also includes openings in its top, intermediate, and bottom portions 26, 23, and 28 enabling a liquid or a gas to be injected into or extracted from the pockets or the intermediate duct 24, either manually or automatically. These openings are preferably fitted with respective septums.

In this example, the access control means 34 are likewise preferably of the “pinch” type, having for this purpose a hollow end which is connected to the end of the link duct 20.

A circuit is thus established between the top bag 6 of the first reservoir 2 and the top bag 27 of the second reservoir 25 via the culture chambers 19-i (i=1 to 3 in this example) and via the link means 20 and 21.

In order to enable culture growth to be controlled thermally, the device has temperature regulation means for regulating temperature inside the culture well(s), or for regulating the temperature of the culture fluid fed to the wells, or indeed, and preferably, for regulating temperature both in the wells and of the culture fluid, as shown in FIGS. 1 to 4.

In the embodiment shown in these figures, the temperature of the culture fluid is regulated in the two reservoirs 2 and 25 by circulating a heat-conveying fluid (liquid or gas) inside their rigid walls 15. More precisely, the walls 15 which define the top and bottom portions of the reservoirs 2 and 25 have fluid circulation spaces 35 integrated therein and forming part of a fluid circuit for heating purposes. As shown in FIGS. 2 to 4, it is advantageous for this circulation space 35 to be defined by assembling together an inner shell 16 and an outer shell 17 housing the inner shell 16.

The inner shell 16 is preferably constituted by assembling together two half-shells 16a and 16b which define the top, intermediate, and bottom inner portions of each reservoir 2 and 25.

The outer shell 17 is likewise preferably constituted by assembling together two half-shells 17a and 17b having first holding means 36 (in this case orifices) for co-operating with second holding means 37 (in this case studs) formed on the outside surface of the inner shell 16. In its top portion it also has an inlet 42 fitted with a first connector 43 (suitable for connection to the “external” main portion of the heating fluid circuit), and in its bottom portion it has an outlet 44 provided with a second connector 45 suitable for being connected to a third or a fourth connector 46 fitted to the end wells 18-1 and 18-3. As a result, the heat-conveying fluid can circulate inside the walls 15 of the reservoirs 2 and 25 and provide effective temperature regulation for the culture fluid which circulates in the bags.

In order to provide temperature regulation in the wells, channels 47 are provided forming another portion of the heating fluid circuit. When the wells 18 are made in a thick solid block 48, the channels 47 are preferably formed by making hollows in said block 48 at the periphery of the zones defining the wells, and preferably also beneath them. In a variant, when the wells and the reservoirs are installed on a support plate, the support plate may include channels 47 for circulating a fraction of the heat-conveying fluid beneath the wells 18. The channels 47 are connected to one side of a third connector 46 for connecting to the second connector 45 of the first reservoir 2 and to the opposite side of the fourth connector 46 for connection to the first connector 43 of the second reservoir 25.

The heat-conveying fluid circulates in the main portion of the heating fluid circuit and thus reaches the walls 15 of the first reservoir 2 via the first connector 43, circulates in the inter-shell space 35, and then reaches the second connector 45. It then penetrates into the channels 47 of the wells 18 via the third connector 46 and reaches the fourth connector 46. Thereafter it penetrates into the walls of the second reservoir 25 via its second connector 45, circulates in the inter-shell space, and then reaches its first connector 43 from which it returns to the main portion of the heating fluid circuit.

In order to enable the heat-conveying fluid to circulate, the main portion of the fluid circuit includes firstly a main container 49 containing a fraction of the heat-conveying fluid and including electric heater means 51, e.g. adjustable heater resistances, an inlet 52 connected via a duct 53 to the first connector 43 of the second reservoir 25, and an outlet 54 connected to a pump (not shown) which feeds the first connector 43 of the first reservoir 2 via another duct 55. This other duct 55 is preferably fitted with two parallel-connected solenoid valves (or pneumatic valves) for regulation purposes, 55 and 57, and with a pressure sensor 58 (or pressure contact). The temperature of the heat-conveying fluid in the main container 49 is selected in such a manner as to ensure that the culture fluid in the outlet 12 of the bottom bag 7 housed in the first reservoir 2 is at a temperature which is suitable for culture purposes.

Naturally, the temperature inside the wells can be different from or substantially identical to the temperature of the culture fluid leaving the first reservoir, depending on requirements.

In a variant embodiment, both reservoirs 2 and 25 and the wells 18 may be fed in parallel with the same heat-conveying fluid, or with heat-conveying fluids coming from two or three independent heating fluid circuits. It is also possible to provide a heating-fluid circuit for each portion of a reservoir. This makes it possible to provide reservoirs containing culture mediums placed at different temperatures on either side of the culture chamber, so as to create temperature profiles or temperatures cycles.

The supplies (or nutrient container) 14 and/or the gas or fluid feed devices (or waste vessels) 32 may also possess their own thermostat circuits so as to maintain their respective contents at selected temperatures which might optionally be different. The thermostatically controlled temperatures may involve heating or cooling. It is generally preferable to maintain them at temperatures lying in the range about 3° C. to about 12° C. in order to ensure that the culture medium is stable.

In another variant, which is completely different, the temperature regulation means comprise electric heater means such as heater resistances or controlled temperature profile (CTP) elements such means may be placed at selected locations on or in the walls defining the reservoirs and/or the wells.

It is also possible to envisage combining heater resistances and a heating fluid circuit.

The power of the electric heater means and/or the flow rate of the heat-conveying fluid is/are controlled by a control unit 50 so as to govern the temperature of the heat-conveying fluid.

Furthermore, in order to improve temperature control in the wells and/or in the reservoirs, one or more temperature sensors may be provided at selected locations to deliver temperature measurements to the control unit.

In order to govern the inside volumes of the top bags 6 and 27 and of the bottom bags 7 and 29, the device of the invention includes pressurization means which are described below with reference to FIG. 1.

In the embodiment shown, pressurization mans are used which are common to two reservoirs 2 and 25, which means are housed in an external unit 105 (such as the unit represented by dashed lines in FIG. 1) together with most of the temperature regulation means. However, in a variant, each reservoir could have its own pressurization means housed in respective units placed, for example, beneath the top portions of the reservoirs.

The pressurization means comprise a high pressure pressurization circuit 59 having a pressure booster (or pump) 60 fed with ambient air 61 and feeding a pressurized supply 62, preferably coupled to a pressure sensor (or pressure contact) 63. The reserve 62 feeds a main duct 64 fitted with a pressure regulator 65 and then a first flow rate regulator 66 and a particle filter 67 (e.g. having a 0.01 micron grid). When the device is for use with a plurality of different pressurization fluids (e.g. air and carbon dioxide), an auxiliary duct 68 is provided which is fed with auxiliary fluid(s) 69 (e.g. carbon dioxide), having a second pressure regulator 70 followed by a second flow rate regulator 71 and feeding the main duct 64 between the first pressure regulator 65 and the filter 67. Under such circumstances, it is advantageous to provide a third flow rate regulator 72 between the filter 67 and the point where the auxiliary duct 68 is connected.

The main duct 64 serves to feed pressurized fluid to the two reservoirs 2 and 25 and also to the culture fluid container 14. In order to minimize temperature disturbances which might be generated by the pressurization fluid on penetrating into the top and bottom portions of the reservoirs 2 and 25, it is heated by means of the heat-conveying fluid that is located in the main container 49. To do this, a portion 73 of the main duct 64 is housed in the main container 49, preferably in the form of a coil therein or in any form that encourages heat exchange.

In addition, in order to be able to control the humidity of the pressurization fluid before it penetrates into the reservoirs 2 and 25, an auxiliary container 74 is preferably provided in the main container 49 and partially filled with a humidifying liquid, the portion 73 of the main duct that is immersed in the heat-conveying fluid opening out into said auxiliary container.

The portion 75 of the main duct 65 which opens out into the auxiliary container 74 feeds, preferably via a thermometer-hygrometer 76, firstly a first port 77 at high pressure (e.g. about 45 millibars (mbar)) which is fitted with four valves 78, 79, 80, and 81 connected in parallel, secondly a second port 82 at high pressure (e.g. about 45 mbar) which opens out into the culture fluid container 14, thirdly a third port 83 at low pressure (e.g. about 10 mbar) which is fitted with four valves 84, 85, 86, and 87 connected in parallel, preferably together with a fourth flow rate regulator 88 placed upstream from the valves, and fourthly a fourth port 89 at intermediate pressure (e.g. about 30 mbar) which is preferably fitted with a fifth flow rate regulator 90 followed by a solenoid valve 91 (or a pneumatic valve) and a pressurized supply 92 feeding in parallel the four valves 78, 79, 80, and 81 which are preferably solenoid valves or pneumatic valves.

In a variant, pressurization fluid circuits may be provided that are different in order to govern the volumes of the bags housed inside the top and bottom portions of the same reservoir. This can make it possible to use different pressurization fluids within the same reservoir so that the bags perform different functions, for example in order to perform comparative tests.

The various solenoid valves (or pneumatic valves) 78-81 and 84-87 are preferably all of the three-port type (two inlets and one outlet), the outlets of the solenoid valves (or pneumatic valves) 78-81 feeding respective ones of the inlets of the solenoid valves (or pneumatic valves) 84-87 whose outlets act respectively via connectors 93-96 connected to the connectors 39, 41 installed in the leaktight inlets 38, 40 to feed the insides of the top and bottom portions 3 and 4 of the first reservoir 2 and of the top and bottom portions 26 and 28 of the second reservoir 25 so as to govern the volumes of the flexible bags contained therein.

These solenoid valves (or pneumatic valves) may also be used for governing the states of the access control means 11, 13, 31, and 34 of the wells 18 and the flexible bags, which, as mentioned above, are preferably of the “pinch” type and are, for example, as described in patent document FR 00/00548. However that is merely one possibility amongst others, and switches or valves could also be used.

All of the solenoid valves and the pressurization fluid pumps are controlled by the electronic control unit 50 which is provided for this purpose with microprocessors (or a microcontroller) mounted on a card which is preferably connected to a link interface 97 (e.g. of the RS232 type) in order to enable it to be remotely controlled by a process computer.

Once programmed, the microcontroller 50 controls the solenoid valves (or pneumatic valves) in such a manner as to apply high and/or low pressure sequences to the bags by means of the pressurization fluid, in accordance with the requirements and in the top and bottom portions 3 and 4 of the reservoirs 2 and 25. Naturally, the microcontroller 50 may include a memory 98, preferably a re-writable memory, containing a multiplicity of culture programs, each culture program defining first and second pressure sequences for governing the respective volumes of the various flexible bags, and also the regulated temperatures of the wells and/or of the heat-conveying fluid.

As mentioned above, instead of using a microcontroller for governing a single pressurization circuit, it is possible to use the same microcontroller to govern two pressurization circuits that are at least partially independent, e.g. installed beneath the top portions of the reservoirs. In another variant, it is possible to use two independent microcontrollers that have previously been synchronized in order to govern two independent pressurization circuits.

The device preferably includes a cover for isolating the well(s) and possibly also the reservoirs from the external medium. This serves not only to avoid exchanges taking place through the various septums, but also to limit temperature disturbances. This also serves to establish a “mechanical” protective barrier around the wells. The cover can also cover the entire device, thereby forming an enclosure defining a biological barrier which is particularly useful when said device is not itself placed under a laminar flow hood. The shape of the cover and the material from which it is made can be selected so as to enable the cells and tissue contained in the wells to be observed under a microscope or using any other suitable optical means while they are being cultured. For this purpose, the cover is preferably made of a material that is not breakable, and that is transparent over the wells.

An outlet for connection to atmospheric pressure may also be provided in the top and bottom portions of the reservoirs 2, 25 being fitted with a solenoid valve (or a pneumatic valve) 99-102 under the control of the control unit 50. In addition, as shown in FIGS. 2 to 4, the inlets 9, 30 of the top flexible bags 6, 27 are preferably placed in a rigid duct 103 defined by the rigid walls of the inner half-shells 16a and 16b and are provided with respective top cavities 104 fitted with draining means (not shown) so as to evacuate any microbubbles of air that might form in operation in the flexible bags of the reservoirs 2 and 25.

The device of the invention can be considered as comprising a control unit coupled with “consumable” type elements (reservoirs and/or wells) that are possibly for single use only. This can be achieved merely by providing the outer control, pressurization, and temperature regulation unit 105 with first and second connection means 93-96, 106 connected respectively to the pressurization and temperature regulation circuits, and secondly the two reservoirs 2 and 25 of each device with third and fourth connection means 39, 41, and 43 respectively connected to the top and bottom inside portions of the reservoirs and to the inter-shell space 35, and then connect the first connection means 93-96 to the third connection means 39, 41 and the second connection means 106 to the fourth connection means 43.

In order to start a new culture, the used consumables are disconnected (the reservoirs and/or wells) and they are replaced with new consumables which are connected to the external control unit.

As shown diagrammatically in FIGS. 5 and 6, it is possible to place a multiplicity of devices 1 in parallel so as to constitute an installation for culturing cells and tissue, either for high throughput (identical cultures) or else for a high degree of differentiation (with different cultures). In this example, the installation has four parallel devices 1-1 to 1-4, each device 1-i (in this case i=1 to 4) having three culture wells 18-j (in this case j=1 to 3) connected in series. The reservoirs with respective heat-conveying fluid circulation spaces are connected to one another, for example, by fitting studs 37 on the inner half-shells 16b through suitable holes 108 formed in the outer half-shells 17a and 17b (see FIG. 6).

These devices can be completely independent from one another. Under such circumstances, they may either have a common control unit which controls pressurization and temperature regulation circuits that are independent from one another, or else independent control units each controlling a single pressurization circuit and a single temperature regulation circuit. Under such circumstances, the regulation temperatures and/or the pressurization fluids can differ from one device to another. However such devices may also depend on one another because some of their wells may be in communication.

It is also possible to envisage an installation in which the devices have wells that are independent from one another, sharing a common pressurization circuit and a common temperature regulation circuit controlled by a common (or main) control unit. Under such circumstances, the major portion of the pressurization means and of the temperature regulation means, and also the main control unit are housed in an external unit 105 (such as that represented by dashed lines in FIG. 1). As a result, it is possible to form an installation in which the devices constitute modular elements of the “consumable” type, possibly for single use only. This can be achieved merely by providing the outer control, pressurization, and temperature regulation unit 105 with first and second connection means 93-96, 106 connected respectively to the pressurization and temperature regulation circuits, and secondly the two reservoirs 2 and 25 of each device with third and fourth connection means 39, 41, and 43 respectively connected to the top and bottom inside portions of the reservoirs and to the inter-shell space 35, and then connect the first connection means 93-96 to the third connection means 39, 41 and the second connection means 106 to the fourth connection means 43.

To proceed with new cultures, the used consumables (reservoirs and/or wells) are removed and replaced by new consumables whose wells have optionally been inoculated with cells.

In such an installation, the number of devices connected in parallel can vary depending on requirements.

In an installation of the invention, as in a device of the invention, the culture wells 18-j may be connected in series on a support plate 107 as shown in FIG. 5, or else they may be formed directly by hollowing out a thick solid block 48 (as shown in FIG. 1).

In the first example (FIG. 5), the support plate 107 may have housings for receiving each of the wells 18-i-j (in this case i=1 to 4 and j=1 to 3) and channels 47 for circulating a fraction of the heat-conveying fluid close to the peripheries of the wells. The support plate 107 may also have channels or ducts for circulating a fraction of the pressurization fluid. In the second embodiment, the culture wells of the devices may be made in independent solid blocks or in a single block. Details concerning embodiments of wells suitable for use in a device of the invention are given in patent document FR 00/00548.

As mentioned above when describing the device 1, it is advantageous to provide a main cover so as to isolate the wells from the outside, and possibly also the two reservoirs of each device of the installation, or indeed all of the devices. This makes it possible to avoid using respective covers for each of the devices.

Examples of implementation (in other words first and second sequences of pressures for governing the volumes of the bags of the reservoirs) of the device and the installation of the invention are to be found in patent document FR 00/00548. It is merely recalled herein that the installation and the device are suitable for operating in the various modes mentioned below.

A “laminar” mode consists in causing the culture fluid to rise into the top bag of one of the two reservoirs so as to establish a difference in height between the top bag and the bottom bags of the two reservoirs, and then in allowing the culture fluid to flow under gravity from the top reservoir towards the bottom reservoirs, and cause the culture fluid to rise towards the top bag of the other reservoir. The same operations are then repeated in the opposite direction (the “return direction”) in order to perform one complete cycle (“go-and-return”) between the two reservoirs via the wells. The number of cycles is selected as a function of the type of culture to be performed in the wells 18. The four steps of a go-and-return cycle in laminar mode are grouped together in FIG. 7. The number of successive cycles is selected as a function of the type of culture to be performed.

A “turbulent” mode consists in applying high pressure continuously to the bottom bags 7 and 29 of the first and second reservoirs 2 and 25. In other words, the first and second sequences of the top bags of the first and second reservoirs are constituted by a succession of four low pressure periods. This mode has only two steps which are grouped together in the form of a “go-and-return” cycle in FIG. 8. The number of successive cycles is selected as a function of the type of culture performed. This mode may be implemented in a first variant (FIG. 9) in which the high pressure is not maintained continuously on the two bottom bags 7 and 29, but on the two top bags 6 and 27. This enables culture fluid to be caused to flow very quickly between the two bottom bags 7 and 29, given that said fluid can no longer rise because of the high pressures in the top bags 6 and 27. In a second variant (not shown), the first sequence applied to each bag of the first reservoir consists in alternating first periods of high pressure with second periods of low pressure, and the second sequence applied to each bag of the second reservoir consists in alternating first periods of low pressure and second periods of high pressure.

The two above-described modes of operation, laminar and turbulent, and also the variant mode, are merely a few of the numerous examples that can be envisaged. Thus, it is possible to combine turbulent operation cycles with laminar operation cycles.

The invention applies to very many types of cells and tissue, such as, in particular:
cells of the intestine: intestine 407, Caco-2, Colo 205, T84, SW 1116, WiDr, HT 29, HT 115, HT 55;
endothelial cells: human aortic smooth muscle cells (HAOSMC);
epidermal cells: human epidermal keratinocyte neonatal (NHEK-Neopooled), Equine Dermis;
cancer cells: HeLa, CHO-K1;
intestine type fibroblast cells: CCD-18Co;
fibroblast cells of MRC-5, 3T3, Wi-38 type;
myelomas: SP20-Ag14, P3X63 Ag8 653, MPC11;
hybridomas;
insect cells: SF9.

This list is not exhaustive in any way; it merely gives examples.

The invention is not limited to the modes of operating the device and the installation as described above merely by way of example, and on the contrary covers all variants that the person skilled in the art might imagine within the ambit of the following claims.

Thus, in the above a temperature regulation circuit is described in which a heat-conveying fluid is circulated for the purpose of raising temperature. However, it is possible to make use also of an auxiliary temperature regulation circuit in which the heat-conveying fluid that circulates serves to remove heat in order to refrigerate certain media, for example the reserves. Naturally, under such circumstances, the device of the invention needs to be fitted with cooling means under the control of the control module.

Furthermore, in the description above, the wells are placed at first selected temperatures and/or the fluid(s) and one or more second selected temperatures. However, it is possible to envisage placing the well(s) under first temperature cycle(s) or profile(s) and/or the fluids(s) under second temperature cycle(s) or profile (s).

In addition, it is also possible to regulate the inlet section of each reservoir and of the chamber, particularly when they are fed by a common heat-conveying circuit, so as to control their respective temperatures independently.

Finally, the temperature regulation means may be arranged in such a manner as to impart a thermal shock to the inside of the chamber and/or the wells. This can be particularly advantageous when it is necessary to modify the state of cell membranes. The thermal shock may be combined with a change in pressure achieved by controlling the flow rate of the fluid and/or the internal pressure of the chamber.

1. A device for culturing cells and tissue, the device being of the type comprising at least one culture well arranged to define a chamber suitable for receiving cells or tissue to be cultured, first and second reservoirs each housing at least one flexible bag, at least one of the bags of the reservoirs being suitable for receiving a culture fluid, link means coupled to said well and to said bags to enable the culture fluid to flow from one reservoir to the other via said well, pressurization means arranged to apply to the bags of the first and second reservoirs respective first and/or second sequences of external pressures defined by at least one control module for causing the culture fluid to flow through said well, the device being characterized in that it includes temperature regulation means controlled by said control module and arranged to maintain a first selected temperature or a first selected temperature cycle inside said well and/or to subject the culture fluid leaving at least one of said first and second reservoirs in order to feed said well to a second selected temperature or to a second selected temperature cycle.

2. A device according to claim 1, characterized in that said temperature regulation means comprise a fluid circuit including a first portion integrated in the walls defining said well and arranged to enable a heat conveying fluid to circulate therethrough.

3. A device according to claim 2, characterized in that the first portion of the fluid circuit includes first connection means opening out into circulation channels for the heat-conveying fluid that are integrated in the walls of the well.

4. A device according to claim 1, characterized in that said temperature regulation means comprise a fluid circuit including second and third portions respectively integrated in the walls defining the first and second reservoirs and arranged to allow a heat-conveying fluid to circulate therethrough.

5. A device according to claim 4, characterized in that each of the first and second reservoirs is made by assembling together an inner shell and an outer shell housing the inner shell and defining an inter-shell space in which the heat-conveying fluid can circulate.

6. A device according to claim 4, characterized in that the second portion of the fluid circuit is arranged to feed heat-conveying fluid to the first portion, and the third portion of said fluid circuit is arranged to collect the heat-conveying fluid that has circulated through the first portion.

7. A device according to claim 6, characterized in that the second and third portions of the fluid circuit include second and third connection means opening out into the inter-shell space and suitable for being connected for a first sub-portion to the first connection means and for a second sub-portion to a fourth portion of the fluid circuit for feeding and collecting heat-conveying fluid.

8. A device according to claim 4, characterized in that said fluid circuit includes a pump coupled to a main container containing a fraction of the heat-conveying fluid and electric heater means for heating said heat-conveying fluid in controlled manner before it is fed to the first, second, and third portions.

9. A device according to claim 1, characterized in that said temperature regulation means comprise first and second electric heater elements for providing at least some of the controlled heating of the well.

10. A device according to claim 1, characterized in that said temperature regulation means include second electric heater elements for providing at least a portion of the controlled heating of the first and second reservoirs.

11. A device according to claim 9, that said electric heater elements comprise heater resistances secured to walls defining the reservoirs and/or the well.

12. A device according to claim 1, characterized in that each of the first and second reservoirs comprises a top portion and a bottom portion interconnected via a narrow intermediate portion, each top and bottom portion of the first and second reservoirs housing a respective flexible bag, said top and bottom flexible bags communicating with each other via the intermediate portions, and said link means communicating with the bottom bags, and the top and bottom portions of the first and second reservoirs each further including a leaktight inlet, and in that the pressurization means comprise a fluid pump suitable for feeding high pressure pressurization fluid via a first portion of the pressurization circuit to a second portion of the pressurization circuit that is connected to top and bottom valves controlled by the control module and suitable for feeding each top and bottom portions of the first and second reservoirs via said leaktight inlets with pressurization fluid at a pressure that is high, low, or intermediate.

13. A device according to claim 12, characterized in that the first portion of the pressurization circuit includes a sub-portion immersed in the heat-conveying fluid contained in the main container in such a manner as to feed the second portion of the pressurization circuit with heated pressurization fluid.

14. A device according to claim 13, characterized in that said pressurization means include an auxiliary container placed in said main container in contact with the heat-conveying fluid, the container containing a humidifying fluid and being fed with pressurization fluid by the sub-portion of the first portion of the pressurization circuit so that the pressurization fluid which feeds the second portion of the pressurization circuit presents a selected degree of humidity.

15. A device according to claim 1, characterized in that it includes at least two wells placed in series and communicating with each other via said link means, one of the wells being connected to said first reservoir and the other well being connected to said second reservoir.

16. A device according to claim 15, characterized in that it includes a third well placed in series between the other two wells and communicating with each of them via said link means.

17. A device according to claim characterized in that the control module includes a memory of the re-writable type suitable for storing said pressure sequences and said first and second selected temperatures, or the first and second selected temperature cycles.

18. A device according to claim 1, characterized in that said control module is arranged to control the inlet sections of each reservoir and of the chamber when they are fed by a common heat-conveying circuit, so as to control their respective temperatures independently.

19. A device according to claim 1 characterized in that it includes at least one nutrient container and gas or fluid feed devices connected to thermostat circuits, and in that said control module is arranged to control said thermostat circuits in such a manner as to maintain the respective contents of the nutrient container and/or of the gas feed devices at selected temperatures.

20. A device according to claim 1, characterized in that each reservoir portion is connected to a heating fluid circuit so that the portions of the reservoirs that are placed on either side of the chamber can be placed at different temperatures so as to create temperature cycles or profiles in said wells.

21. A device according to claim 1, characterized in that said temperature regulation means are arranged to impart a temperature shock to the inside of the chamber and/or the wells.

22. A device according to claim 1, characterized in that the temperature regulation means include at least one temperature sensor suitable for supplying the control module with measurements representative of the temperature inside a well.

23. A device according to claim 1 characterized in that it includes a cover for isolating at least the wells from the outside.

24. A device according claim 23, characterized in that the cover is for isolating both the wells and the reservoirs from the outside.

25. An installation for culturing cells and tissue, characterized in that it comprises at least two devices according to claim 1, placed in parallel, together with a main control unit for controlling said devices together.

26. An installation according to claim 25, characterized in that it includes a main fluid circuit feeding the wells and/or reservoirs of each device in parallel.

27. An installation according to claim 25, characterized in that it includes central temperature regulation means controlled by said main control unit and arranged to maintain the same selected first temperature or the same selected first temperature cycle inside the wells of each device and/or for subjecting the culture fluid that leaves at least one of said first and second reservoirs of each device for feeding its wells to the same selected second temperature or to a same selected second temperature cycle.

28. An installation according to claim 27, characterized in that said main control unit controls the temperature regulation means of each device so that they maintain independently of one another a first selected temperature or a first selected temperature cycle within the wells of the device and/or so that they act independently of one another to subject the culture fluid that leaves at least one of said first and second reservoirs of the device for feeding its wells to a second selected temperature or to a second selected temperature cycle.

29. An installation according to claim 25, characterized in that it includes a main cover for isolating at least the wells of each device simultaneously from the outside.

30. An installation according to claim 29, characterized in that said main cover serves to isolate the wells and the reservoirs of each device simultaneously from the outside.
